SEQUENCE LISTING (6 sequences)

SEQ ID NO: 1 - DNA, 1033 bp, Arabidopsis thaliana
gaattcgtggtatagcgttacttaataacaattataaactgtaaaatataaatattttataaaaataaaatttgcaagttttaatatatattatttttaaaaataaatcgtcccgcgatataccgcgggttaaaatctagtttctttttgtttatgtaacatcaatagaggtaatctaattaccattcatttaaaataccaaaaataggggaaaaaatgttcttcgttggaatccaattgttccacacaaatttcacatatacaaattaatttaatttacatacaagtggccgacaatatgagattttattcgacaaatcttttatataacctcatcattcacaatttaatagtaaaatagtacgttaatatatcaaccttttcaagcattatggtattctccatccttagattcaaaaacatatgcctgaaatattagatctaacggttgatagtaatagagtgctgtacactcggatctactagaatatctctccaagaccaaccacgagtaaagtaaacgcatcaagtcaagtttttgatgattagagtctttctttatttcaaaagtatatccaagtagattagtgttgtatagattacgtcatcattgtgacatcatctcgtaaccgcttcatgtcacgttgtcatcccaaacacatcatcatctgacgtaattttaacgttgatatctccgcgttcttttactcttttacccctggtcccttttttaacaattataaaaattatgattactattttgaatttacagggaaccaaaaaacccatttataaagtttcgaaattaaatatcctaactaacttggaaaaatatctccaattttcgttttttgtggtttaaattgtaactaggaaactaaaaggactaaagagtaaaagaggtggggacagggtgttttagtctttttctaagaagtttcgtgatgactaatttttttcctttaaaagaaagtcatcagacgaaaaggattatttgccttcttcactctccaaaatcaatcactttctctaa

SEQ ID NO: 2 - DNA, 1226 bp, Arabidopsis thaliana
gatctctatgtatataaaaatatgggtaatattagaaactaactatgaaatggaaaagaattgagagaatgacattgtgtcagaaaagttaggtaaataacattttctgaaaaagagaaaatacaaaaatatccttgtgtttacttatttttacaataatgccattggctttagttataaagtttatatgtatttgtctaaaatagcatgatatatttacaaaaatcatgcaattctttaaaatacatacagaatatatatacacgatatatatgtttctctgaaataatgtgtttctcagaaatagcacgaaatatttataaaaagcatgcaattctcttatagatcgcgaagtttaaaaaaacatatagaattgttacaatattacatgggtttttattggataacatgacaaatatttatttatttcatgagtttttattggatagcatgacaaatattaatatatcagtgttaataacatgttttgttcttaaaatacatgcattttaaaatcagacatttgttttaaaatcaaatctaatctcttatatcacaacgacattgacggaaaattcaggtaaaaagagaaaataaagaatgagagatagagagatttctatggaaaaagaaagagagaacatgtaggtgaacaaaataaagagatatgatgatatattttatgagaggtggtgaagattattttaggagagggagagagaaatagaaaaagaaaatgacatggtgaatctgaagaagatgaattgtgttaaagatgaagagagaaagagaactccatggctaaagtctcgtaaagaagatgaaaaagaaacaaaagaaggaagaagaaagagaaaggctaaaatagactaactattgccaaaatttctgtagccgacaaatactatttggtccaaggttattttgtgtattcttttgaagtcaaaagttatttcttacatatactctaaaaatatagccgataccaatttttccacacatggacttcctttattccaaaagtcaataaagtgtgacgtcatgatacttacgctttaaaacatcgcatgatgatgtcattagcatcaatctccaccgtccaatttatttagttgttgacaatatcgaccgtctaagttccacaccgacggctataagagtttcattataaattttagcaaaataaaatcagcaaataattttttcttgactaagcttaaacgacg

SEQ ID NO: 3 - DNA, 32 bp, Artificial sequence
  Primer N1-P1 for the amplification of the NIMIN-1 gene
ccaagcttgtctcatgaattcgtggtatagcg

SEQ ID NO: 4 - DNA, 31 bp, Artificial sequence
  Primer N1-P2 for the amplification of the NIMIN-1 gene
ccggatccttagagaaagtgattgattttgg

SEQ ID NO: 5 - DNA, 21 bp, Artificial sequence
  Primer N2-P1 for the amplification of the NIMIN-2 gene
ccccacgttaacgatgatcac

SEQ ID NO: 6 - DNA, 28 bp, Artificial sequence
  Primer N2-P2 for the amplification of the NIMIN-2 gene
ctggatcccgtcgtttaagcttagtcaa

(19) United States
(12) Patent Application Publication
(10) Pub. No.: US 2006/0242728 A1
(43) Pub. Date: Oct. 26, 2006

(54) Chemically-inducible promotors for the expression of proteins in plants

(76) Inventors: Artur Pfitzner, Stuttgart (DE); Bernhard Roth, Stuttgart (DE)

Correspondence Address: WEINGARTEN, SCHURGIN, GAGNEBIN & LEBOVICI LLP, TEN POST OFFICE SQUARE, BOSTON, MA, 02109, US

(21) Appl. No.: 10/566,201

(22) PCT Filed: Jul. 27, 2004
(86) PCT No.: PCT/EP04/08405

§ 371 Date: Jan. 27, 2006

(30) Foreign Application Priority Data
Jul. 28, 2003 (EP) 03017114.4

(51) Int. Cl. A01H 1/00; C12N 15/82; C12N 5/04

(52) U.S. Cl. 800/278; 435/419; 435/468

(57) ABSTRACT

The present invention relates to nucleic acids containing selectively inducible regulatory sequences, particularly promoters, for a controlled expression of desired expression products in suitable host expression organisms such as transgenic plants. Further, the present invention relates to a method for detecting the activity of a regulatory sequence in suitable cells, wherein a transgene comprises a potential regulatory sequence operably linked to the Bax gene or a functional derivative thereof and the Bax expression correlates with the activity of the regulatory sequence in said cells.

The present invention relates to nucleic acids containing selectively inducible regulatory sequences, particularly promoters, for a controlled expression of desired expression products in suitable host expression organisms such as transgenic plants. Further, the present invention relates to a method for detecting the activity of a regulatory sequence in suitable cells, wherein a transgene comprises a potential regulatory sequence operably linked to the Bax gene or a functional derivative thereof and the Bax expression correlates with the activity of the regulatory sequence in said cells.

With reference to industrial and medical applications (e.g. enzymes for technical uses, therapeutical or diagnostic antibodies, vaccines) the expression of proteins in transgenic plants is becoming more and more interesting for pharmaceutical and chemical companies. The major reasons are security and costs. Traditionally, valuable proteins for pharmaceutical applications (hormones, antibodies etc.) are produced in cultivated animal cells. Since most cultivation media contain 10% or more fetal calf serum (FCS), there is always the danger of contamination with BSE causing agents. In addition, animal tissue cultures can be contaminated with viruses which may be transmitted to humans. Furthermore, because of the FCS and other additives, the cultivation media are often very expensive. Additional costs come from the high energy requirement for temperature control of large cultivation entities. This is also true for the production of recombinant proteins in microorganisms.

Cultivation of plants is cheap because only sun light, water and some inorganic nutritions are necessary to support plant growth. In addition, plants can be grown on fields in almost unlimited quantity. Therefore, there are no scale-up problems.

However, one essential requirement for the successful production of a heterologous protein in a transgenic organism is the combination of the respective transgene with strong and reliable regulatory elements, the promoters. Promoters lead to an efficient synthesis of the transgenic mRNA, which is then translated to the protein.

For the production of recombinant proteins in transgenic plants the 35S RNA promoter is used most frequently (Pietrzak et al. (1987) Nucleic Acids Res. 14:5857). The 35S promoter was originally isolated from the cauliflower mosaic virus (CaMV) and leads to a high and constitutive synthesis of a transcript for the desired genes. Other promoters, which are less frequent in use are the 19S RNA promoter from CaMV (Paszkowski et al. (1985) Mol. Gen. Genet. 199:178) and the promoter from the nopaline synthase gene from Agrobacterium tumefaciens (An et al. (1984) EMBO J. 4:277).

All these promoters are active during the whole life of a plant and therefore the recombinant protein is synthesized all the time. If these proteins continuously accumulate during the development of the plant, they can be degraded or modified and accordingly lose their biological activity. In most cases, the expressed protein is harmful or even lethal to the particular plant. This can be a major obstacle in establishing transgenic plants with high expression levels. In this case, it is not possible to obtain any transgenic plant at all, or only plants which show very low levels of protein expression.

Therefore, it would be highly desirable to possess a promoter which can be switched on specifically at any time of the plant development by treatment of the plant with chemicals. Transgenic plants could be grown to an appropriate size and then the expression of the recombinant protein could be induced to a high level for a short time. In that way problems like protein degradation or killing of the plants by the heterologous protein could be avoided.

The most prominent plant promoters which can be induced by chemicals are the promoters of genes encoding pathogenesis-related (PR) proteins. PR genes are induced during the plant's defence reaction against pathogens. They are switched off most of the time during the development of the plant and they can be induced by the plant hormone salicylic acid. The major problem with these promoters comes from the fact that they are also induced by a developmental stimulus shortly before flowering of the plants (Grüner and Pfitzner (1994) Eur. J. Biochem. 220:247). Therefore, it is hardly possible to get seed material from plants expressing proteins which are harmful to the particular organism.

Furthermore, for the protein production in transgenic plants it is necessary to know in which parts of the plants the regulatory regions, in particular promoters, of the transgenes are active and inducible, respectively. In order to analyse the regulatory regions in living plants, it is necessary to express reporter genes under the control of these regulatory regions. At present, the reporter genes GUS (β-Glucoronidase), Luc (luciferase), and GFP (green fluorescent protein) are used in plants. However, these reporter genes are not very sensitive and the analysis of whole plants over the complete lifetime is not possible. Further, in contrast to animal cells, plant cells possess a rigid cell wall. Therefore, the substrate application for the reporter genes GUS and Luc is difficult, defective, takes a lot of time and is expensive. Moreover, the use of GFP in plants is difficult due to their autofluorescence. At present, no reporter gene is available to detect sensitively, easily, and over the whole lifetime of a plant the activity of regulatory regions. Thus, the technical problem underlying the present invention is to provide nucleic acids containing regulatory sequences, particularly for plants, which can be selectively switched on by externally added chemicals thereby inducing the expression of transgenes, wherein the regulatory sequences should not be switched on during the normal development of e.g. the plant. A further object of the present invention is to provide a method for detecting the activity of a regulatory sequence in plants or parts thereof, wherein the regulatory activity is easily detectable over the whole lifetime of the plant.

The solution to the above technical problems is achieved by the embodiments characterized in the claims.

In particular, the present invention relates to a recombinant nucleic acid containing at least a first nucleotide sequence operably linked to at least a second nucleotide sequence containing a transgene to be expressed, wherein the first nucleotide sequence contains a regulatory sequence selected from the group consisting of SEQ-ID-No. 1, SEQ-ID-No. 2, and a biologically active derivative thereof. The term “recombinant nucleic acid” is based on the fact that said nucleic acid contains a transgene, and therefore, is obtained by recombinant DNA technology.

The term “biologically active derivative” means any nucleotide sequence with substantially the same biological function as the nucleotide sequences SEQ-ID-Nos. 1 or 2. The biologically active derivative may contain deletions, additions and/or substitution of bases, wherein these alterations do not have substantial influence on the regulating activity of said derivatives when compared to SEQ-ID-Nos. 1 or 2.

The first nucleotide sequence comprises a regulatory sequence, in particular a promoter sequence. The term “regulatory sequence” includes a nucleotide sequence containing elements necessary for the expression/transcription of a desired product such as e.g. a transgene. The regulatory sequence may be regulatory elements which do not constitutively express e.g. a gene but are selectively inducible by chemicals resulting in an expression of e.g. a gene upon induction. The wording “no constitutive expression” means that the regulatory sequence is preferably completely inactive prior to any measure resulting in an induction thereof. In a preferred embodiment of the present invention, the first nucleotide sequence is an inducible plant promoter originating from sequences associated with NIMIN (non-inducible immunity interacting) genes.

In a preferred embodiment of the present invention the chemicals are selected from the group consisting of organic compounds. More preferably the chemicals are selected from the group consisting of phenolic compounds, thiamine, benzoic acid, isonicotinic acid (INA), and derivatives thereof. The phenolic compounds are for example salicylic acid or structural or functional related derivatives thereof.

In a preferred embodiment of the present invention the first nucleotide sequence is derived from Arabidopsis thaliana.

In the recombinant nucleic acid of the present invention the above defined first nucleotide sequence is operably linked to a second nucleotide sequence containing a transgene to be expressed. There is no particular limitation of the transgene contained in the second nucleotide sequence to be expressed, and upon expression/transcription in a suitable host organism the transgene results in e.g. a polypeptide, a protein, or a RNA molecule.

The term “operably linked” means that the first and second nucleotide sequences are connected to each other in such a way that the regulatory sequence contained in the first nucleotide sequence controls the transcription/expression of the transgene contained in the second nucleotide sequence.

The transgene contained in the second nucleotide sequence of the recombinant nucleic acid of the present invention is expressed upon induction by exogenous stimuli, e.g. chemicals. Surprisingly, the expression of the transgene contained in the second nucleotide sequence is substantially not induced by any endogenous stimuli during the whole lifetime of a transgenic plant containing the recombinant nucleic acid of the present invention.

In a preferred embodiment of the present invention the second nucleotide sequence of the recombinant nucleic acid contains further a reporter system for the analysis of expression patterns. This reporter system may be any reporter system known in the art comprising nucleotide sequences, wherein the expression/transcription of said nucleotide sequences results in a detectable signal. The signal may be detected on a molecular level, e.g. by measuring the increase of a particular mRNA via Northern-Blot or RT-PCR or by the analysis of differences in the expression level of a particular polypeptide or protein e.g. by a Western-Blot analysis. Alternatively the signal may be detected on a macroscopic level, e.g. by detecting visual alterations of the phenotype of a host organism containing said recombinant nucleic acid. Examples for said reporter systems are those comprising the reporter genes GUS (β-Glucoronidase), Luc (luciferase), and GFP (green fluorescent protein).

The reporter system for the analysis of expression patterns according to the present invention may be suitable for in vitro-applications, e.g. in a desired cell culture, or in vivo-applications, e.g. In a desired host organism.

In a further preferred embodiment of the present invention the reporter system contains an element which causes apoptosis/necrosis of the transformed cell upon transcription of the second nucleotide sequence. The induction of apoptosis/necrosis may be detected by any method known in the art and/or commercially available. The induction of apoptosis/necrosis may be detected on a molecular level, e.g. by detecting DNA fragmentation or by detecting the up-regulation of the expression of specific peptides and/or proteins, or on a cellular level. These detection methods may include procedures which require a specific pretreatment of apoptotic/necrotic cells, e.g. for FACS analysis or the detection of specifically stained apoptotic/necrotic cells using a microscope, as well as procedures which do not require any pre-treatment of the apoptotic/necrotic cells, e.g. the visual detection of necrotic plant cells in a leaf. In a more preferred embodiment of the present invention the reporter system contains the human Bax gene or a biologically active derivative thereof.

The present invention also relates to vectors comprising the above-defined recombinant nucleic acid according to the invention. Examples of vectors are pBin19, pGC4-00, pUC18/19, pBluescript.

The present invention also relates to a host organism, preferably a non-human host organism, comprising the recombinant nucleic acid according to the invention or the vector according to the invention. The host organism is for example a prokaryotic organism such as E. coli or Agrobacterium tumefaciens, or an eukaryotic organism such as a plant cell (including a transgenic plant). The recombinant nucleic acid according to the present invention can be also contained in a virus or viroid suitable for the preparation of a transgenic organism such as a transgenic plant.

Further, the present invention relates to the use of the above-defined recombinant nucleic acid for the preparation of a transgenic plant whose transgene is selectively inducible by exogenously added chemicals. The term “exogenously added chemicals” includes chemicals that are not produced by the plant itself (or produced only in amounts which do not substantially cause induction of the above-defined regulatory sequence), and chemicals that are produced by the plant at some time during the lifetime. The term “selectively inducible” means that the expression of the transgene is substantially induced (“switched on”) only by adding specific chemicals.

The present invention further relates to a transgenic plant such as a tobacco plant, tomato plant, or potato plant comprising the recombinant nucleic acid according to the present invention for the purpose of a regulated expression of any homologous or heterologous transgene. The term “transgenic plant” includes the whole plant as such and parts thereof, such as root, stem, leaf, organ-specific tissue or cells, the reproductive material thereof, especially seeds, and the seedlings thereof. The term “heterologous transgene” means a gene which derives from a source other than the wildtype of the transgenic plant. The term “homologous transgene” means a gene which derives from the wildtype of the transgenic plant. In one embodiment of the present invention the transgenic plant contains the above-defined recombinant nucleic acid of the invention stably integrated into the genetic material. Alternatively, the transgenic plant expresses transiently the transgene included in the second nucleotide sequence which is contained in the above-defined recombinant nucleic acid.

In a preferred embodiment of the present invention the expression of the above-defined transgene contained in the second nucleotide sequence is induced after treatment with chemicals of the transgenic plant. The chemicals are preferably selected from the group consisting of organic compounds as defined above.

Surprisingly, the first nucleotide sequence of the recombinant nucleic acid according to the present invention is capable to induce selectively the expression of a desired sequence (“transgene”) in e.g. transgenic plants after treatment with certain chemicals. Accordingly, it is possible to grow the transgenic plants to a size sufficient to obtain enough plant material, and then to induce the expression/transcription of a transgene resulting in the desired product such as e.g. a polypeptide, a protein or a RNA molecule. In contrast to the regulatory sequences most frequently used as inducible plant promoters in the state of the art, which are also inducible by endogenous stimuli like e.g. stress factors, the regulatory sequences contained in the recombinant nucleic acid of the present invention are substantially inducible only by exogenously added chemicals. Thus, the present invention provides a tool allowing the highly regulated expression of a desired transgene over the whole lifetime of a host organism such as a plant, which may be combined with a highly sensitive and easily detectable reporter system.

The recombinant nucleic acid according to the present invention and the vector according to the present invention can be prepared by means of methods known in the art. This includes also methods for cultivating suitable host cells and recovering said recombinant nucleic acids or said vectors from said hosts cells and/or the culture medium.

The present invention also relates to a method using the human Bax gene as an analytical tool for the analysis of expression patterns of a regulatory sequence in suitable host organisms such as cells or even whole living plants. Following activation of the regulatory sequences by chemicals the distinct expression pattern of the regulatory sequence is detectable by the new in vivo reporter system, the human Bax gene. In this context “in vivo” means in a living organism, for example a plant or a part thereof. Further, the method is also applicable for analysing the potential of chemicals to activate/induce certain regulatory sequences.

The method for detecting the activity of a regulatory sequence in suitable cells comprises
(a) preparing transformed cells, comprising at least a nucleotide sequence coding for the Bax gene or a biologically active derivative thereof, operably linked to a nucleotide sequence comprising a potential regulatory sequence,
(b) treating the transformed cells with a chemical,
(c) measuring the expression of the Bax gene or the biologically active derivative thereof in the transformed cells, and
(d) correlating the Bax expression with the activity of the regulatory sequence.

The term “biologically active derivative” within the context of the Bax gene means any nucleotide sequence with substantially the same biological function as the Bax gene.

The term “regulatory sequence” is as defined above. In a preferred embodiment of the present invention the regulatory promoter sequence induces the expression of the Bax gene, upon treatment with chemicals. The chemicals are preferably as defined above.

According to the method of the present invention it is possible to detect the activity of a regulatory sequence by inducing gene expression. The expression of the Bax gene correlates with the activity of the regulatory sequence. The method according to the present invention is useful for analysing both the activity of the regulatory sequence as well as the potential of chemicals to modulate the activity of the regulatory sequence. Further, it is possible to study the activity of a regulatory sequence during the whole development of a suitable host organism such as a plant. The Bax protein effectively induces apoptosis/necrosis in the cell. Thus, when using plants as a suitable host organism, the activity of the regulatory sequence to be analysed is simply visible as necrotic areas on the plant. The activity of the regulatory sequence to be analysed can also be detected by any other method known in the art for the detection of apoptosis/necrosis as outlined above.

The figures show:

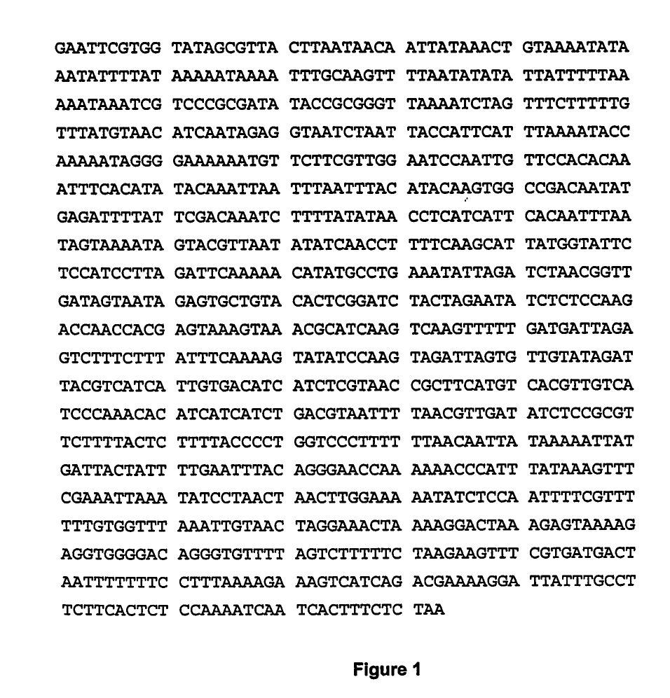

FIG. 1 shows the nucleotide sequence of the NIMIN-1 promoter (SEQ-ID-No.1).

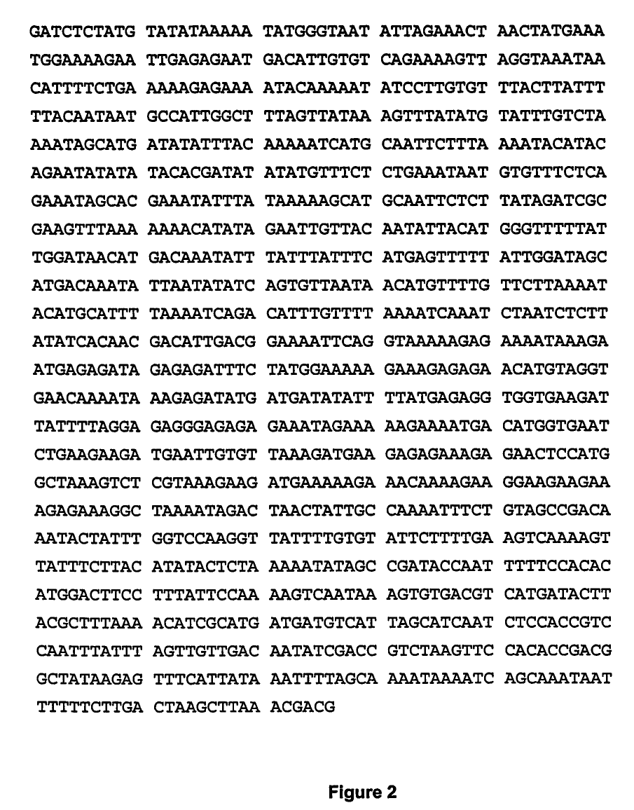

FIG. 2 shows the nucleotide sequence of the NIMIN-2 promoter (SEQ-ID-No.2).

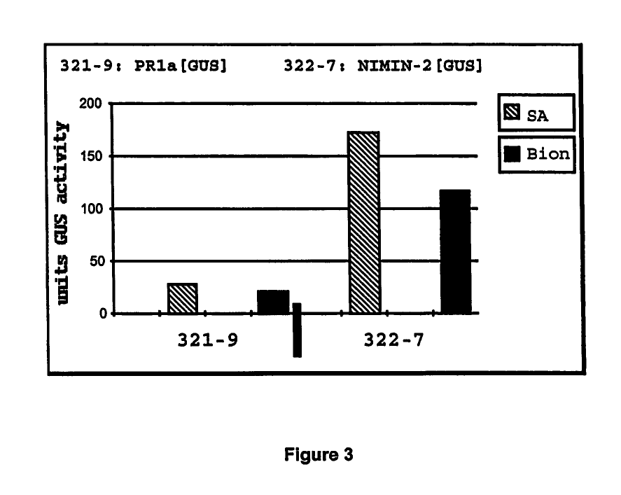

FIG. 3 shows a bar diagram comparing the GUS (beta-glucoronidase) activity of PR1a-GUS (321-9) and NIMIN2-GUS (322-7) fusion constructs in transgenic plants after induction with salicylic acid (SA) or Bion, wherein the GUS-activity of water-treated fusion-constructs in transgenic plants is used as background control.

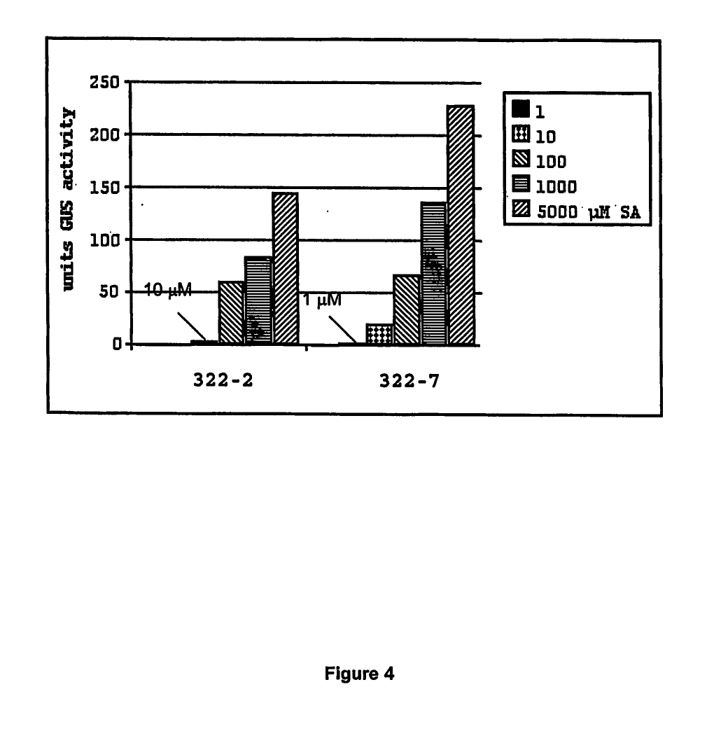

FIG. 4 shows a bar diagram which correlates the amount of SA used for the treatment of the plant with the GUS activity in two independent NIMIN2-GUS transgenic plants (322-2 and 322-7) after induction with salicylic acid (SA), wherein the GUS-activity of water-treated fusion-constructs in transgenic plants is used as background control.

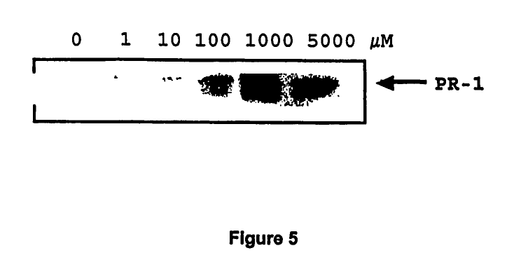

FIG. 5 shows a Western Blot which correlates the amount of SA used for the treatment of the plant with the expression of the endogenous PR-1 proteins in transgenic plant 322-2 as depicted in FIG. 4, and which is used as a positive control.

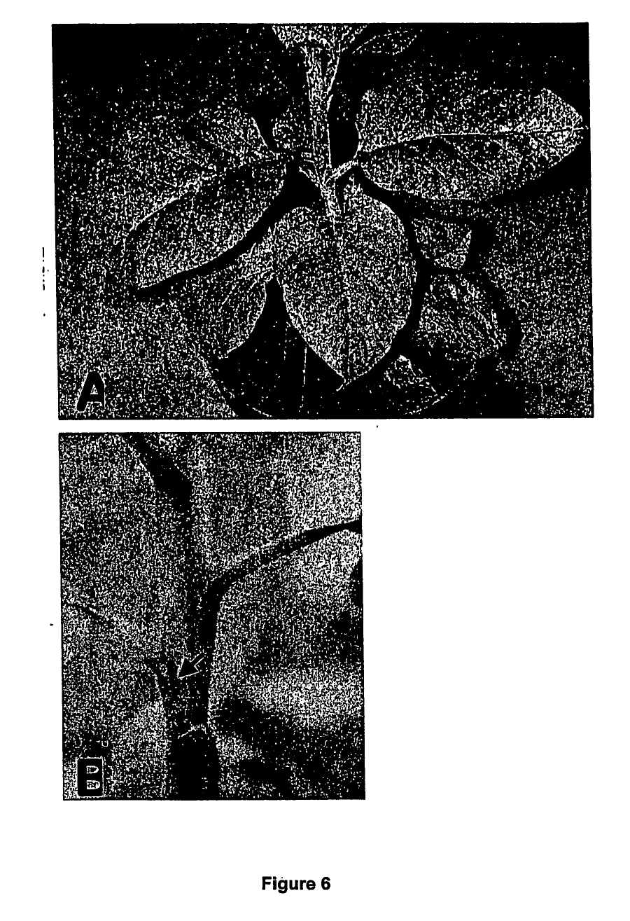

FIG. 6 shows the spontaneous expression of the Bax gene under the control of the PR-1 a promoter (A) in leafs and (B) on the stem of transgenic tobacco plants.

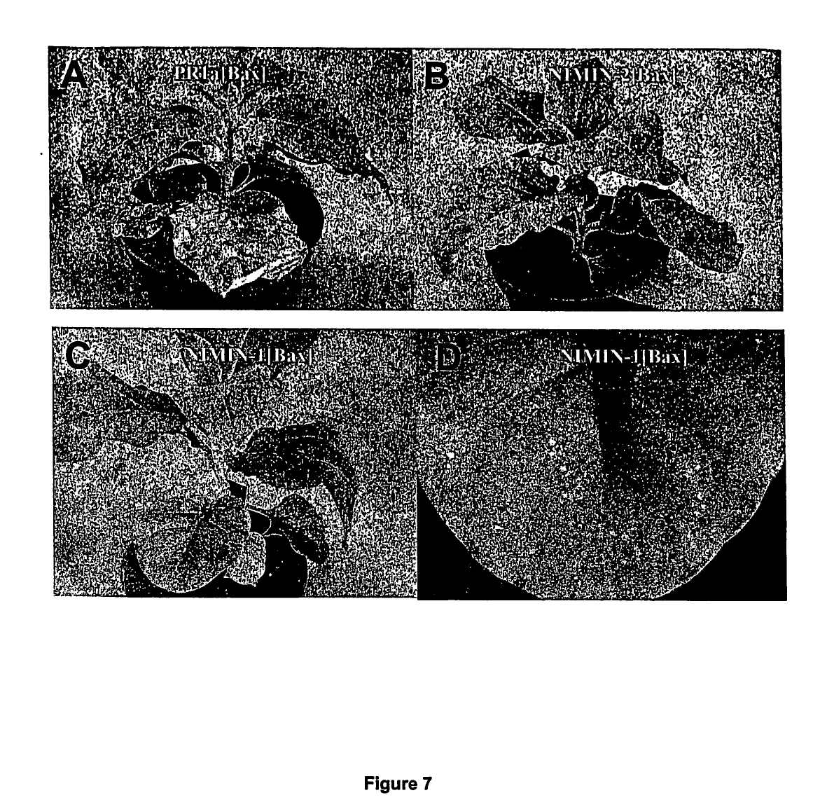

FIG. 7 shows plants which contain the human Bax gene under the control of the PR-1a promoter (A), the NIMIN-2 promoter (B) or the NIMIN-1 promoter (C and D).

The present invention will now be further illustrated in the following examples, without being limited thereto.

### EXAMPLES

### Example 1

### Isolation of the NIMIN-1 and NIMIN-2 Promoters

The cDNAs for the NIMIN proteins NIMIN-1, NIMIN-2 and NIMIN-3 have been isolated as new interacting proteins with the regulatory NPR1 protein in Arabidopsis thaliana (Weigel et al. (2001) Plant Mol. Biol. 46:143). The mRNAs for the NIMIN-1 and NIMIN-2 proteins were shown to be inducible by salicylic acid and Bion, a substance which can replace salicylic acid in some of its functions (Weigel et al. (2001) Plant Mol. Biol. 46:143). To identify the regulatory elements responsible for this induction, the promoter elements were amplified by PCR from genomic DNA of Arabidopsis thaliana using the primers N1-P1 (SEQ-ID-No. 3) and N1-P2 (SEQ-ID-No. 4) for NIMIN-1 and N2-P1 (SEQ-ID-No. 5) and N2-P2 (SEQ-ID-No. 6) for NIMIN-2 (N1-P1: 5′-CCAAGCTTGTCTCATGAATTCGTGGTATAGCG-3′; N1-P2: 5′-CCGGATCCTTAGAGAAAGTGATTGATTTTGG-3′; N2-P1: 5′-CCCCACGTTAACGATGATCAC-3′; N2-P2: 5′-CTGGATCCCGTCGTTTAAGCTTAGTCAA-3′). The respective PCR fragments were analysed on an agarose gel and yielded the expected sizes (1.1 kb for NIMIN-1 and 1.2 kb for NIMIN-2). The fragments were cut with the restriction enzymes HindIII and BamHI (NIMIN-1 promoter element) or BgIII and BamHI (NIMIN-2 promoter element), and ligated in the cloning vector pUC19. The resulting clones were sequenced and the nucleotide sequence is shown in FIG. 1 (NIMIN-1) and FIG. 2 (NIMIN-2).

### Example 2

### Construction of Reporter Gene Fusions and Generation of Transgenic Plants

The DNA fragments containing the respective promoter sequences were excised from the pUC19 subclones and ligated in pUC/0-GUS (Beilmann et al. (1991) Eur. J. Biochem. 196:415). The resulting ligation products were analysed by a restriction enzyme digest with EcoRI (NIMIN1-GUS) or XbaI and EcoRI (NIMIN2-GUS). The 3.2 kb band, representing the reporter gene-promoter fusion was excised from an agarose gel and ligated in the Agrobacterium vector pBin19, yielding pBin19/N1-GUS and pBin19/N2-GUS. The plasmids were moved from E. coli into Agrobacterium tumefaciens via triparental mating.

The resulting Agrobacteria strains were used to generate transgenic plants containing the reporter gene constructs.

### Example 3

### Characterization of the Promoter Activities of the NIMIN Promoters

For characterizing the influence of chemicals on the activity of the NIMIN promoters, leaf discs were punched from the transgenic lines NIMIN1-GUS and NIMIN2-GUS. As a control, PR-1a promoter fusions were used. The leaf discs were floated on 1 mM SA, 140 mg/l Bion (a functional analogue to salicylic acid) or water. After 3 days, protein extracts were prepared and the activity of the GUS reporter gene in the extracts was assayed. The resulting reporter gene activities were 5 times higher In the NIMIN-2 promoter fusions than In PR-1a promoter fusions (cf. FIG. 3). Further, leaf discs from the transgenic lines NIMIN1-GUS and NIMIN2-GUS were incubated with different salicylic acid concentrations. The activity of the NIMIN promoters depends on the salicyclic acid concentration (cf. FIGS. 4 and 5).

### Example 4

### Construction of a Reporter Gene (Bax) Fusion with the PR-1 a Promoter and Generation of Transgenic Tobacco Plants

The vector pUC19/His(Bax) was digested with the restriction enzymes SacI and BgIII. The resulting DNA fragment containing the respective human Bax alpha gene with an N-terminal 6× His tag was ligated in the binary vector pBin19/PR1a-GUS (Beilmann et al. (1992) Plant Mol. Biol. 18:65), in which the GUS was excised before with restriction enzymes SacI and BamHI. The ligation product was transformed in E. coli DH5α, and the resulting colonies were analyzed by a restriction enzyme digest with EcoRI and HindIII and PCR, yielding the vector pBin19/PR1a-Bax. The plasmid was moved from E. coli to Agrobacterium tumefaciens LBA4404 via electroporation. The resulting Agrobacterium strain was used to generate transgenic tobacco plants (Nicotiana tabacum var. Samsun NN/nn) containing the reporter gene construct.

### Example 5

### Analysis of the Regulation of the NIMIN Promoters by Endogenous Stimuli

The most important feature of an inducible promoter is not only the inducibility by exogenous substances but also that the promoter is switched off under normal conditions. To Investigate, if the NIMIN promoters fulfil these requirements, transgenic tobacco plants with NIMIN promoter-Bax fusions were generated as described above. Bax is lethal for any cell. Therefore, any leakiness of the NIMIN promoter should lead to necrotic areas in the plants. Plants were grown in the green house and monitored for their phenotype.

While use of the PR-1 a promoter resulted in major necrotic regions later in the development of the plants (cf. FIG. 6 and FIG. 7A), there were no necrotic areas on the NIMIN2-Bax plants and only minor lesions on the lower leafs of NIMIN1-Bax plants (cf. FIG. 7B-D) showing that the NIMIN promoters are significantly less leaky, i.e. switched on by endogenous stimuli, than the PR-1a promoter.

1. A recombinant nucleic acid containing at least a first nucleotide sequence operably linked to at least a second nucleotide sequence containing a transgene to be expressed, wherein the first nucleotide sequence contains a regulatory sequence selected from the group consisting of SEQ-ID-No. 1, SEQ-ID-No. 2, and a biologically active derivative thereof.

2. The recombinant nucleic acid according to claim 1, wherein the regulatory sequence is a promoter sequence selectively inducible by chemicals.

3. The recombinant nucleic acid according to claim 2, wherein the chemicals are selected from the group consisting of organic compounds.

4. The recombinant nucleic acid according to claim 3, wherein the organic compounds are selected from the group consisting of phenolic compounds, thiamine, benzoic acid, isonicotinic acid (INA), and derivatives thereof.

5. The recombinant nucleic acid according to claim 4, wherein the phenolic compound is salicylic acid or a structural or functional derivative thereof.

6. The recombinant nucleic acid according to claim 1, further containing a reporter system which comprises at least one nucleotide sequence, wherein the expression/transcription of said nucleotide sequence results in a detectable signal.

7. A vector containing the recombinant nucleic acid according to claim 1.

8. A host organism containing the recombinant nucleic acid according to claim 1.

9. The host organism according to claim 8, which is selected from the group consisting of a bacteria cell and a plant cell.

10. A transgenic plant containing at least the recombinant nucleic acid according to claim 1.

11. The transgenic plant according to claim 10, wherein the recombinant nucleic acid is stably integrated into the genetic material.

12. The transgenic plant according to claim 10, wherein the transgene contained in the second nucleotide sequence is transiently expressed.

13. The transgenic plant according to claim 10, wherein the expression of the transgene contained in the second nucleotide sequence is selectively induced upon treatment with chemicals.

14. The transgenic plant according to claim 13, wherein the chemicals are selected from the group consisting of organic compounds.

15. A method for detecting the activity of a regulatory sequence in suitable cells, comprising
(a) preparing transformed cells, comprising at least a nucleotide sequence coding for the Bax gene or a biologically active derivative thereof, operably linked to a nucleotide sequence comprising a potential regulatory sequence,
(b) treating the transformed cells with a chemical,
(c) measuring the expression of the Bax gene or the biologically active derivative thereof in the transformed cells, and
(d) correlating the Bax expression with the activity of the regulatory sequence.

16. The method according to claim 15, wherein the regulatory sequence is a promoter sequence.

17. (canceled)

18. The method according to claim 15, wherein the transformed cells form at least part of a transgenic plant.

19. The method according to claim 15, wherein the expression of the Bax gene is detected as necrotic area in the plant.

20. A host organism containing the vector according to claim 7.

21. The host organism according to claim 20, which is selected from the group consisting of a bacteria cell and a plant cell.

22. The transgenic plant according to claim 11, wherein the transgene contained in the second nucleotide sequence is transiently expressed.

23. The transgenic plant according to claim 11, wherein the expression of the transgene contained in the second nucleotide sequence is selectively induced upon treatment with chemicals.

24. The transgenic plant according to claim 23, wherein the chemicals are selected from the group consisting of organic compounds.

25. The recombinant nucleic acid according to claim 5, further containing a reporter system which comprises at least one nucleotide sequence, wherein the expression/transcription of said nucleotide sequence results in a detectable signal.

26. A vector containing the recombinant nucleic acid according to claim 25.

27. A host organism containing the recombinant nucleic acid according to claim 25.

28. A host organism containing the vector according to claim 26.

29. A method for detecting the activity of a regulatory sequence in suitable cells, comprising
(a) preparing transformed cells, comprising at least a nucleotide sequence coding for the Bax gene or a biologically active derivative thereof, operably linked to a nucleotide sequence comprising a potential regulatory sequence,
(b) treating the transformed cells with a chemical selected from the group of claim 3,
(c) measuring the expression of the Bax gene or the biologically active derivative thereof in the transformed cells, and
(d) correlating the Bax expression with the activity of the regulatory sequence.

30. A method for detecting the activity of a regulatory sequence in suitable cells, comprising
(a) preparing transformed cells, comprising at least a nucleotide sequence coding for the Bax gene or a biologically active derivative thereof, operably linked to a nucleotide sequence comprising a potential regulatory sequence,
(b) treating the transformed cells with a chemical selected from the group of claim 5,
(c) measuring the expression of the Bax gene or the biologically active derivative thereof in the transformed cells, and
(d) correlating the Bax expression with the activity of the regulatory sequence.

31. The method according to claim 30, wherein the transformed cells form at least part of a transgenic plant.

32. The method according to claim 31, wherein the expression of the Bax gene is detected as necrotic area in the plant.

33. The host organism according to claim 20, which is selected from the group consisting of a bacteria cell and a plant cell.
